(19) United States
(12) Patent Application Publication
(10) Pub. No.: US 2005/0153403 A1
(43) Pub. Date: Jul. 14, 2005

(54) Process for preparing L-amino acids using strains of the enterobacteriaceae family

(76) Inventor: Nicole Dusch, Werther (DE)

Correspondence Address: FITCH, EVEN, TABIN & FLANNERY, P. O. BOX 65973, WASHINGTON, DC, 20035, US

(21) Appl. No.: 11/017,120

(22) Filed: Dec. 21, 2004

Related U.S. Application Data
(60) Provisional application No. 60/607,362, filed on Sep. 7, 2004.

(30) Foreign Application Priority Data
Dec. 24, 2003 (DE) 103 61 268.8

(51) Int. Cl. C12P 13/04; C12N 1/21

(52) U.S. Cl. 435/106; 435/252.3; 435/252.33

(57) ABSTRACT

The invention relates to a process for preparing L-amino acids by fermenting recombinant microorganisms of the Enterobacteriaceae family. The microorganisms are characterized by the overexpression or enhancement of the yaaU ORF. The desired L-amino acid is isolated, with, optionally, constituents of the fermentation broth, and/or the biomass remaining in the isolated product.

### CROSS REFERENCE TO RELATED APPLICATIONS

The present application claims priority to German application DE 103 61 268.8, filed on Dec. 24, 2003, the contents of which is hereby incorporated by reference. In addition, the present application claims the benefit of U.S. provisional application 60/607,362, filed on Sep. 7, 2004.

### FIELD OF THE INVENTION

This invention relates to a process for preparing L-amino acids, especially L-threonine, using recombinant microorganisms of the Enterobacteriaceae family in which the open reading frame (ORF) designated yaaU is enhanced. The invention also includes the microorganisms themselves.

### BACKGROUND OF THE INVENTION

L-amino acids, especially L-threonine, are used in human medicine, in the pharmaceutical industry, in the foodstuff industry and in animal nutrition. These amino acids can be prepared by fermenting Enterobacteriaceae strains, e.g., Escherichia coli and Serratia marcescens. Because of the economic importance of L-amino acids, efforts are continually being made to improve the methods by which they are prepared. Such improvements may relate to: fermentation technology, e.g., methods of stirring or supplying oxygen; the composition of the nutrient media, e.g., the sugar concentration during the fermentation; the processing or purification of the product formed, e.g., by means of ion exchange chromatography; or the intrinsic performance properties of the microorganism itself.

Methods of mutagenesis, and selection are often used to improve the performance of microorganisms. These methods may result in strains which are resistant to antimetabolites, such as the threonine analog α-amino-β-hydroxyvaleric acid (AHV), or that are auxotrophic for metabolites of regulatory importance.

For a number of years, recombinant DNA methods have also been used for improving L-amino acid-producing strains of the Enterobacteriaceae family. Such methods generally involve amplifying individual amino acid biosynthesis genes and investigating the effect that such amplification has on production. Information on the cell biology and molecular biology of Escherichia coli and Salmonella can be found in Neidhardt (ed): Escherichia coli and Salmonella, Cellular and Molecular Biology, 2^{nd} edition, ASM Press, Washington, D.C., USA, (1996).

### OBJECT OF THE INVENTION

The objective of the present invention is to provide new improved fermentation methods for the preparation of L-amino acids, particularly L-threonine.

### BRIEF DESCRIPTION OF THE DRAWINGS

FIG. 1: Map of the yaaU gene-containing plasmid pTrc99AyaaU.

### DESCRIPTION OF THE INVENTION

The invention relates to recombinant microorganisms of the Enterobacteriaceae family which contain an enhanced or overexpressed yaaU-ORF, encoding a polypeptide that is a putative sugar transporter. These microorganisms produce L-amino acids, especially L-threonine, in an improved manner. In each case, microorganisms which are not recombinant for the yaaU-ORF, and which do not contain an enhanced yaaU-ORF are used as the starting point for comparison.

The recombinant microorganisms include microorganisms of the Enterobacteriaceae family in which a polynucleotide is enhanced that encodes a polypeptide whose amino acid sequence is at least 90%, preferably at least 95%, more preferably at least 98%, still more preferably 99%, still more preferably 99.7% and most preferably 100%, identical to an amino acid sequence selected from the group SEQ ID NO:4, SEQ ID NO:6 and SEQ ID NO:8. The microorganisms preferably contain an enhanced or overexpressed polynucleotide selected from the group:
a) a polynucleotide having the nucleotide sequence of SEQ ID NO:3, SEQ ID NO:5 or SEQ ID NO:7;
b) a polynucleotide having a nucleotide sequence which corresponds to SEQ ID NO:3, SEQ ID NO:5 or SEQ ID NO:7 within the limits of the degeneracy of the genetic code;
c) a polynucleotide sequence having a sequence which hybridizes, under stringent conditions, with the sequence which is complementary to the sequence SEQ ID NO:3, SEQ ID NO:5 or SEQ ID NO:7;
d) a polynucleotide having the sequence of SEQ ID NO:3, SEQ ID NO:5 or SEQ ID NO:7 which contains functionally neutral sense mutants.

In each case the polynucleotides encode a putative sugar transporter.

The invention also relates to a process for fermentatively preparing L-amino acids, especially L-threonine, using recombinant microorganisms of the Enterobacteriaceae family which, preferably, already produce L-amino acids and in which at least the open reading frame (ORF) having the designation yaaU, or nucleotide sequences encoding its gene product, is or are enhanced. Preferred microorganisms are the ones described herein.

As used herein, the term “L-amino acids” or “amino acids” refers to one or more amino acids, including their salts, selected from the group L-asparagine, L-threonine, L-serine, L-glutamate, L-glycine, L-alanine, L-cysteine, L-valine, L-methionine, L-proline, L-isoleucine, L-leucine, L-tyrosine, L-phenylalanine, L-histidine, L-lysine, L-tryptophan, L-arginine and L-homoserine. L-threonine is particularly preferred.

The term “enhance” describes the increase, in a microorganism, of the intracellular activity or concentration of one or more enzymes or proteins which are encoded by the corresponding DNA. For example, enhancement may be accomplished: by increasing the copy number of the gene or genes, or of the ORF or ORFs by at least one (1) copy; by using a strong promoter operatively linked to the gene; or by using a gene or allele, or an ORF which encodes a corresponding enzyme or protein having high activity. Where appropriate, these measures may also be combined.

An open reading frame (ORF) is a segment of a nucleotide sequence which encodes, or can encode, a protein and/or a polypeptide or ribonucleic acid and for which the prior art is unable to assign any function. After a function has been assigned to the nucleotide sequence segment in question, this segment is generally referred to as a “gene.” Alleles are generally understood as being alternative forms of a given gene. The forms are distinguished by differences in the nucleotide sequence. In general, the protein, or the ribonucleic acid, encoded by a nucleotide sequence, i.e., an ORF, a gene or an allele, is designated as a “gene product.”

Enhancement measures, in particular overexpression, generally increase the activity or concentration of the corresponding protein by at least 10%, 25%, 50%, 75%, 100%, 150%, 200%, 300%, 400% or 500%, maximally up to 1000% or 2000%, relative to wild-type protein or the activity or concentration of the protein in the nonrecombinant parent strain or microorganism. The non-recombinant microorganism or parent strain is understood as being the microorganism on which the measures according to the invention are performed.

In one aspect, the invention relates to a process for preparing L-amino acids by fermenting recombinant microorganisms of the Enterobacteriaceae family, characterized in that:
a) the desired L-amino acid-producing microorganisms, in which the open reading frame yaaU, or nucleotide sequences or alleles encoding the gene products thereof, is/are enhanced, in particular overexpressed, are cultured in a medium under conditions in which the desired L-amino acid is enriched in the medium or in the cells, and
b) the desired L-amino acid is isolated, optionally with the fermentation broth constituents and/or the biomass remaining in its/their entirety or in portions (from ≧0 to 100%) in the isolated product or being removed completely.

The microorganisms with an enhanced or overexpressed yaaU open reading frame which are in particular recombinant, are likewise part of the subject matter of the present invention. They can produce L-amino acids from glucose, sucrose, lactose, fructose, maltose, molasses, starch and cellulose or from glycerol and ethanol. The microorganisms are representatives of the Enterobacteriaceae family and are selected from the genera Escherichia, Erwinia, Providencia and Serratia. The genera Escherichia and Serratia are preferred with the most preferred species being Escherichia coli or Serratia marcescens.

In general, recombinant microorganisms are generated by means of transformation, transduction or conjugation, or a combination of these methods, with a vector which contains the desired ORF, the desired gene, an allele of this ORF or gene, or parts thereof, and/or a promoter which enhances the expression of the ORF or gene. This promoter can be produced by an enhancing mutation in the endogenous regulatory sequence located upstream of the gene or ORF. Alternatively, an efficient promoter may be fused to the gene or ORF in a recombinant vector.

Examples of suitable E. coli strains which produce L-threonine are:
Escherichia coli H4581 (EP 0 301 572)
Escherichia coli KY10935 (Biosci. Biotech. Biochemistry 61(11): 1877-1882 (1997)
Escherichia coli VNIIgenetica MG442 (U.S. Pat. No. 4,278,765)
Escherichia coli VNIIgenetica M1 (U.S. Pat. No. 4,321,325)
Escherichia coli VNIIgenetica 472T23 (U.S. Pat. No. 5,631,157)
Escherichia coli BKIIM B-3996 (U.S. Pat. No. 5,175,107)
Escherichia coli cat 13 (WO 98/04715)
Escherichia coli KCCM-10132 (WO 00/09660)

Examples of suitable L-threonine producing strains of the species Serratia marcescens, are:
Serratia marcescens HNr21 (Appl. Environ. Microbiol. 38(6): 1045-1051 (1979))
Serratia marcescens TLr156 (Gene 57(2-3): 151-158 (1987))
Serratia marcescens T-2000 (Appl. Biochem. Biotechnol. 37(3): 255-265 (1992)).

L-Threonine-producing strains of the Enterobacteriaceae family preferably possess, inter alia, one or more genetic or phenotypic features selected from the group: resistance to α-amino-β-hydroxyvaleric acid; resistance to thialysine; resistance to ethionine; resistance to α-methylserine; resistance to diaminosuccinic acid; resistance to α-aminobutyric acid; resistance to borrelidin; resistance to cyclopentanecarboxylic acid; resistance to rifampicin; resistance to valine analogs such as valine hydroxamate; resistance to purine analogs, such as 6-dimethylaminopurine; a requirement for L-methionine; a possible partial and compensatable requirement for L-isoleucine; a requirement for mesodiaminopimelic acid; auxotrophy in regard to threonine-containing dipeptides; resistance to L-threonine; resistance to threonine raffinate; resistance to L-homoserine; resistance to L-lysine; resistance to L-methionine; resistance to L-glutamic acid; resistance to L-aspartate; resistance to L-leucine; resistance to L-phenylalanine; resistance to L-serine; resistance to L-cysteine; resistance to L-valine; sensitivity to fluoropyruvate; a defective threonine dehydrogenase; an ability to utilize sucrose; enhancement of the threonine operon; enhancement of homoserine dehydrogenase I-aspartate kinase I, preferably of the feedback-resistant form; enhancement of homoserine kinase; enhancement of threonine synthase; enhancement of aspartate kinase, preferably of the feedback-resistant form; enhancement of aspartate semialdehyde dehydrogenase; enhancement of phosphoenolpyruvate carboxylase, preferably of the feedback-resistant form; enhancement of phosphoenolpyruvate synthase; enhancement of transhydrogenase; enhancement of the RhtB gene product; enhancement of the RhtC gene product; enhancement of the YfiK gene product; enhancement of a pyruvate carboxylase; and attenuation of acetic acid formation.

It has been found that, following overexpression of the gene or the open reading frame (ORF) yaaU, or its alleles, microorganisms of the Enterobacteriaceae family produce L-amino acids, in particular L-threonine, in an improved manner. The nucleotide sequences of the Escherichia coli genes or open reading frames (ORFs) belong to the prior art and can be obtained from the Escherichia coli genome sequence published by Blattner et al., (Science 277: 1453-1462 (1997)). It is known that endogenous enzymes (methionine aminopeptidase) are able to cleave off the N-terminal amino acid methionine.

The nucleotide sequences for the yaaU-ORF from Shigella flexneri and Salmonella typhimirium, which likewise belong to the Enterobacteriaceae family, have also been disclosed. The yaaU ORF of Escherichia coli K12 is described, inter alia, by the following data:
Designation: open reading frame
Function: annotated as a putative transport protein, a membrane transporter of the MFS (major facilitator superfamily) family. The transported substrates vary greatly; the MFS family contains monosaccharide, disaccharide and oligosaccharide transporters, potassium and amino acid transporters, transporters for intermediates of the tricarboxylic acid cycle and also transporters which pump antibiotics out of the cells.
Description: the open reading frame yaaU encodes a 48.7 KDa protein; the isolelectric point is 8.8; when located in the chromosome, yaaU is present, for example in the case of Escherichia coli K12 MG1655, in the intergenic region of the genes carB, encoding the long chain of carbamoyl phosphate synthase, and kefC, encoding a (K(+)/H(+)glutathione-regulated potassium efflux system protein;
Reference: Blattner et al.; Science 277(5331): 1453-1474 (1997)
Accession No.: AE000114
Alternative gene name: B0045

The nucleic acid sequences can be obtained from the databases belonging to the National Center for Biotechnology Information (NCBI) of the National Library of Medicine (Bethesda, Md., USA), the nucleic acid sequence database of the European Molecular Biology Laboratories (EMBL, Heidelberg, Germany or Cambridge, UK) or the Japanese DNA database (DDBJ, Mishima, Japan). For the sake of greater clarity, the nucleotide sequence of the Escherichia coli yaaU-ORF is given in SEQ ID NO:3 and the sequences for the yaaU-ORF of Shigella flexneri (AE015041) and Salmonella typhimurium (AE008697) are given under SEQ ID No:5 and, respectively, SEQ ID NO:7. The amino acid sequences of the proteins encoded by these reading frames are depicted as SEQ ID NO:4, SEQ ID NO:6 and, respectively, SEQ ID NO:8.

The open reading frames described in the passages above can be used in accordance with the invention. In addition, it is possible to use alleles of the genes or open reading frames, which result from the degeneracy of the genetic code or as a consequence of functionally neutral sense mutations. Preference is given to using endogenous genes or endogenous open reading frames.

“Endogenous genes” or “endogenous nucleotide sequences” are understood as being the genes or open reading frames or alleles or nucleotide sequences which are present in a species population.

Alleles of the yaaU-ORF, which contain functionally neutral sense mutations, include, inter alia, those which lead to: at most 50; at most 40; at most 30; at most 20; preferably, at most 10; at most 5; and most preferably, at most 3 or at most 2, or at least one, conservative amino acid substitution in the protein which they encode.

In the case of the aromatic amino acids, substitutions are said to be conservative when phenylalanine, tryptophan and tyrosine are substituted for one another. In the case of hydrophobic amino acids, substitutions are said to be conservative when leucine, isoleucine and valine are substituted for one another. In the case of polar amino acids, substitutions are said to be conservative when glutamine and asparagine are substituted for one another. In the case of the basic amino acids, substitutions are said to be conservative when arginine, lysine and histidine are substituted for one another. In the case of the acidic amino acids, substitutions are said to be conservative when aspartic acid and glutamic acid are substituted for one another. In the case of the hydroxyl group-containing amino acids, substitutions are said to be conservative when serine and threonine are substituted for one another.

It is also possible to use nucleotide sequences which encode variants of proteins, which variants additionally contain an extension or truncation by at least one (1) amino acid at the N terminus or C terminus. This extension or truncation amounts to not more than 50, 40, 30, 20, 10, 5, 3 or 2 amino acids or amino acid residues.

Suitable alleles also include those which encode proteins in which at least one (1) amino acid has been inserted or deleted. The maximum number of such changes, termed indels, can affect 2, 3, 5, 10, 20, but, in no case more than 30, amino acids. Suitable alleles also include those which can be obtained by means of hybridization, in particular under stringent conditions, using SEQ ID NO:3, SEQ ID NO:5 or SEQ ID NO:7 or parts thereof, and, in particular, the coding regions or the sequences which are complementary thereto.

Instructions for identifying DNA sequences by means of hybridization may be found in, inter alia, the manual The DIG System Users Guide for Filter Hybridization supplied by Boehringer Mannheim GmbH (Mannheim, Germany, 1993) and Liebl et al. (Intn'l J Systematic Bacteriol. 41: 255-260 (1991)). Stringent conditions may be chosen such that the only hybrids formed are those in which the probe and target sequence, i.e., the polynucleotides treated with the probe, are at least 80% identical. It is known that the stringency of hybridizations, including the washing steps, is influenced and/or determined by buffer composition, temperature and salt concentration. In general, the hybridization reaction is carried out at a stringency which is relatively low as compared with that of the washing steps (Hybaid Hybridization Guide, Hybaid Limited, Teddington, UK, 1996). For example, a buffer corresponding to 5×SSC can be used for the hybridization reaction at a temperature of approx. 50° C.-68° C. Under these conditions, probes can also hybridize with polynucleotides which possess less than 70% identity with the sequence of the probe. These hybrids are less stable and are removed by washing under stringent conditions. This can be achieved, for example, by lowering the salt concentration to 2×SSC and, where appropriate, subsequently to 0.5×SSC (The DIG System User's Guide for Filter Hybridization, Boehringer Mannheim, Mannheim, Germany, 1995) with the temperatures adjusted to approx. 50° C.-68° C., approx. 52° C.-68° C., approx. 54° C.-68° C., approx. 56° C.-68° C., approx. 58° C.-68° C., approx. 60° C.-68° C., approx. 62° C.-68° C., approx. 64° C.-68° C., approx. 66° C.-68° C. Temperature ranges of approx. 64° C.-68° C. or approx. 66° C.-68° C. being preferred. It is possible, where appropriate, to lower the salt concentration down to a concentration corresponding to 0.2×SSC or 0.1×SSC. By means of increasing the hybridization temperature stepwise, in steps of approx. 1-2° C., from 50° C. to 68° C., it is possible to isolate polynucleotide fragments which, for example, possess at least 80%, or at least 90%, at least 91%, at least 92%, at least 93%, at least 94%, at least 95%, at least 96%, at least 97%, at least 98%, or at least 99%, identity with the sequence of the probe employed or with the nucleotide sequences shown in SEQ ID NO:3, SEQ ID NO:5 or SEQ ID NO:7. Additional instructions for hybridizations can be obtained commercially in the form of kits (e.g., DIG Easy Hyb from Roche Diagnostics GmbH, Mannheim, Germany, Catalog No. 1603558). The nucleotide sequences which are thus obtained encode polypeptides which possess at least 90%, preferably at least 95%, more preferably at least 98%, still more preferably 99%, still more preferably 99.7%, identity with the amino acid sequences depicted in SEQ ID NO:4, SEQ ID NO:6 or SEQ ID NO:8.

Enhancement may be achieved, for example, by increasing the expression of the genes or open reading frames or alleles or by increasing the catalytic properties of the protein. Both measures can be combined, where appropriate.

In order to achieve overexpression, the copy number of the corresponding genes or open reading frames can be increased or the promoter region and regulatory region or the ribosome binding site which is located upstream of the structural gene can be mutated. Expression cassettes which are incorporated upstream of the structural gene act in the same manner. It is also possible to increase expression during the course of the fermentative L-threonine production by incorporating inducible promoters; in addition, using promoters for gene expression which permits a different chronological gene expression can also be advantageous. Expression is likewise improved by means of measures for extending the lifespan of the mRNA. Enzyme activity may also be enhanced by preventing protein degradation. The ORFs, genes or gene constructs can either be present in plasmids having different copy numbers or be integrated, and amplified, in the chromosome. Alternatively, overexpression of the genes can be achieved by altering the composition of the media and the conditions of culture.

Methods for overexpression are described in the prior art, for example in Makrides et al. (Microbiol. Rev. 60(3): 512-538 (1996)). Vectors can be used and will increase the copy number by at least one (1) copy. The vectors can be plasmids as described, for example, in U.S. Pat. No. 5,538,873. Vectors can also be phages, for example phage Mu, as described in EP 0332448, or phage lambda (λ). Copy number can also be increased by incorporating an additional copy into another site in the chromosome, for example in the att site of phage λ (Yu, et al., Gene 223: 77-81 (1998)). U.S. Pat. No. 5,939,307 reports that it is possible to increase gene expression by incorporating expression cassettes or promoters, such as the tac promoter, the trp promoter, the lpp promoter, the P_{L} promoter or the P_{R} promoter of phage λ, upstream of the chromosomal threonine operon. In the same way, it is possible to use the phage T7 promoters, the gearbox promoters or the nar promoter. Such expression cassettes or promoters can also be used as described in EP 0 593 792 to overexpress plasmid-bound genes. Using the lacI^{Q} allele makes it possible to control the expression of such genes (Glascock et al., Gene 223: 221-231 (1998)). It is also possible for gene activity to be increased by modifying genomic sequences by means of one or more nucleotide substitutions, insertions or deletions. Altered gene expression can also be achieved, for example, as described in Walker et al. (J. Bacteriol. 181: 1269-80 (1999)), using the growth phase-dependent fis promoter. One of skill in the art can find general instructions in this regard in, inter alia: Chang, et al., J. Bacteriol. 134: 1141-1156 (1978); Hartley, et al., Gene 13: 347-353 (1981); Amann, et al., Gene 40: 183-190 (1985); de Broer et al., Proc. Nat'l Acad. Sci. USA 80: 21-25 (1983); LaVallie, et al., BIO/TECHNOLOGY 11: 187-193 (1993); PCT/US97/13359; Llosa et al., Plasmid 26: 222-224 (1991), Quandt, et al., Gene 80: 161-169 (1989), Hamilton, et al., J. Bacteriol. 171: 4617-4622 (1989), Jensen et al., Biotech. Bioeng. 58: 191-195 (1998) and in textbooks of genetics and molecular biology.

Plasmid vectors which can replicate in Enterobacteriaceae, such as pACYC184-derived cloning vectors (Bartolomé et al., Gene 102: 75-78 (1991)), pTrc99A (Amann et al., Gene 69: 301-315 (1988)) or pSC101 derivatives (Vocke, et al., Proc. Nat'l Acad. Sci. USA 80(21): 6557-6561 (1983)) can be used in the invention. For example, a bacterial strain may be used which is transformed with a plasmid vector carrying at least one nucleotide sequence, or allele, encoding the yaaU ORF or its gene product. The term “transformation” is understood as meaning the uptake of an isolated nucleic acid by a host (microorganism).

It is also possible to use sequence exchange (Hamilton, et al.; J. Bacteriol. 171: 4617-4622 (1989)), conjugation or transduction to transfer mutations which affect the expression of genes or open reading frames, into bacterial strains. More detailed explanations of the concepts of genetics and molecular biology can be found in textbooks of genetics and molecular biology such as the textbook by Birge (Bacterial and Bacteriophage Genetics, 4^{th} ed., Springer Verlag, New York, USA, 2000) or the textbook by Berg, et al. (Biochemistry, 5^{th} ed., Freeman and Company, New York, USA, 2002) or the manual by Sambrook et al. (Molecular Cloning, A Laboratory Manual, (3-Volume Set), Cold Spring Harbor Laboratory Press, Cold Spring Harbor, USA, 2001).

When using strains of the Enterobacteriaceae family to produce L-amino acids, in particular L-threonine, it may be advantageous, in addition to enhancing the open reading frame yaaU, to enhance one or more enzymes of the threonine biosynthesis pathway, enzymes of anaplerotic metabolism, enzymes for producing reduced nicotinamide adenine dinucleotide phosphate, enzymes of glycolysis, PTS enzymes, or enzymes of sulfur metabolism. Endogenous genes are generally preferred for this purpose. Thus, it is possible, for example, to simultaneously enhance, and preferably overexpress, one or more genes selected from the group:
at least one gene of the thrABC operon encoding aspartate kinase, homoserine dehydrogenase, homoserine kinase and threonine synthase (U.S. Pat. No. 4,278,765);
the pyruvate carboxylase-encoding Corynebacterium glutamicum pyc gene (WO 99/18228);
the phosphoenolpyruvate synthase-encoding pps gene (Mol. Gen. Genetics 231(2): 332-336 (1992));
the phosphoenolpyruvate carboxylase-encoding ppc gene (WO 02/064808);
the pntA and pntB genes encoding the subunits of pyridine transhydrogenase (Eur. J. Biochem. 158: 647-653 (1986));
the rhtB gene encoding the homoserine resistance-mediating protein (EP-A-0 994 190);
the rhtC gene encoding the threonine resistance-mediating protein (EP-A-1 013 765);
the threonine export carrier protein-encoding Corynebacterium glutamicum thrE gene (WO 01/92545);
the glutamate dehydrogenase-encoding gdhA gene (Nucl. Ac. Res. 11: 5257-5266 (1983); Gene 23: 199-209 (1983));
the phosphoglucomutase-encoding pgm gene (WO 03/004598);
the fructose biphosphate aldolase-encoding fba gene (WO 03/004664);
the ptsHIcrr operon ptsH gene encoding the phosphohistidine protein hexose phosphotransferase of the PTS phosphotransferase system (WO 03/004674);
the ptsHIcrr operon ptsI gene encoding enzyme I of the PTS phosphotransferase system (WO 03/004674);
the ptsHIcrr operon crr gene encoding the glucose-specific IIA component of the PTS phosphotransferase system (WO 03/004674);
the ptsG gene encoding the glucose-specific IIBC component (WO 03/004670);
the lrp gene encoding the regulator of the leucine regulon (WO 03/004665);
the fadR gene encoding the regulator of the fad regulon (WO 03/038106);
the iclR gene encoding the regulator of central intermediary metabolism (WO 03/038106);
the ahpCF operon ahpC gene encoding the small subunit of alkyl hydroperoxide reductase (WO 03/004663);
the ahpCF operon ahpF gene encoding the large subunit of alkyl hydroperoxide reductase (WO 03/004663);
the cysteine synthase A-encoding cysK gene (WO 03/006666);
the cysB gene encoding the regulator of the cys regulon (WO 03/006666);
the cysJIH operon cysJ gene encoding the NADPH sulfite reductase flavoprotein (WO 03/006666);
the cysJIH operon cysI gene encoding the NADPH sulfite reductase hemoprotein (WO 03/006666);
the adenylyl sulfate reductase-encoding cysJIH operon cysH gene (WO 03/006666);
the rseABC operon rseA gene encoding a membrane protein which possesses anti-sigmaE activity (WO 03/008612);
the rseABC operon rseC gene encoding a global regulator of the sigmaE factor (WO 03/008612);
the sucABCD operon sucA gene encoding the decarboxylase subunit of 2-ketoglutarate dehydrogenase (WO 03/008614);
the sucABCD operon sucB gene encoding the dihydrolipoyltranssuccinase E2 subunit of 2-ketoglutarate dehydrogenase (WO 03/008614);
the suc ABCD operon sucC gene encoding the β-subunit of succinyl-CoA synthetase (WO 03/008615);
the sucABCD operon sucD gene encoding the α-subunit of succinyl-CoA synthetase (WO 03/008615);
the aceE gene encoding the E1 component of the pyruvate dehydrogenase complex (WO 03/076635);
the aceF gene encoding the E2 component of the pyruvate dehydrogenase complex (WO 03/076635);
the rseB gene encoding the regulator of the SigmaE factor activity (Mol. Microbiol. 24(2): 355-371 (1997));
the gene product of the Escherichia coli yodA open reading frame (ORF) (Accession Number AE000288 of the National Center for Biotechnology Information (NCBI, Bethesda, Md., USA; DE10361192.4)).

In addition to enhancing the open reading frame yaaU, it can also be advantageous to attenuate, eliminate or reduce the expression of one or more of the genes selected from the group:
the threonine dehydrogenase-encoding tdh gene (J. Bacteriol. 169: 4716-4721 (1987));
the malate dehydrogenase (E.C. 1.1.1.37)-encoding mdh gene (Arch. Microbiol. 149: 36-42 (1987));
the gene product of the Escherichia coli yjfA open reading frame (ORF) (Accession Number AAC77180 of the National Center for Biotechnology Information (NCBI, Bethesda, Md., USA, (WO 02/29080));
the gene product of the Escherichia coli ytfP open reading frame (ORF) (Accession Number AAC77179 of the National Center for Biotechnology Information (NCBI, Bethesda, Md., USA, WO 02/29080));
the pckA gene encoding the enzyme phosphoenolpyruvate carboxykinase (WO 02/29080);
the pyruvate oxidase-encoding poxB gene (WO 02/36797);
the dgsA gene (WO 02/081721), which is also known under the name mlc gene, encoding the DgsA regulator of the phosphotransferase system;
the fruR gene (WO 02/081698), which is also known as the name cra gene, encoding the fructose repressor;
the rpoS gene (WO 01/05939), also known as the katF gene, encoding the sigma³⁸ factor; and
the aspartate ammonium lyase-encoding aspA gene (WO 03/008603).

In this context, the term “attenuation” describes the reduction or abolition in a microorganism of the intracellular activity or concentration of one or more enzymes or proteins which are encoded by the corresponding DNA, by, for example, using a weaker promoter than in the parent strain, a gene or allele which encodes a corresponding enzyme or protein having a lower activity, or inactivating the corresponding enzyme or protein, or the open reading frame or gene, and, where appropriate, combining these measures. In general, attenuation measures should lower the activity or concentration of the corresponding protein from 0 to 75%, from 0 to 50%, from 0 to 25%, from 0 to 10% or from 0 to 5% of the activity or concentration of the wild-type protein or of the activity or concentration of the protein for the parent strain, i.e., for the microorganism which is not recombinant for the corresponding enzyme or protein. The parent strain or microorganism is understood as being the microorganism on which the measures according to the invention are performed.

In order to achieve attenuation, the expression of genes or open reading frames, or the catalytic properties of the enzyme proteins, can be reduced or abolished. Where appropriate, both of these measures can be combined. Gene expression can be reduced by altering culture conditions, by genetically altering (mutating) the signal structures for the gene or by means of the antisense RNA technique. Signal structures for gene expression are, for example, repressor genes, activator genes, operators, promoters, attenuators, ribosome binding sites, the start codon and terminators. Information in this regard can be found in, inter alia, Jensen, et al., (Biotech. Bioeng. 58: 191-195 (1998)), Carrier, et al., (Biotech. Prog. 15: 58-64 (1999)), Franch et al., (Curr. Opin. Microbiol. 3: 159-164 (2000)) and in textbooks of genetics and molecular biology such as the textbook by Knippers (Molekulare Genetik [Molecular Genetics], 6th edition, Georg Thieme Verlag, Stuttgart, Germany, 1995) or that by Winnacker (Gene und Klone [Genes and Clones], VCH Verlagsgesellschaft, Weinheim, Germany, 1990).

Mutations which lead to a change or reduction in the catalytic properties of enzymes are known from the prior art. Examples are provided in articles by Qiu, et al., (J. Biol. Chem. 272: 8611-8617 (1997)), Yano et al. (Proc. Nat'l Acad. Sci. USA 95: 5511-5515 (1998)) and Wente et al., (J. Biol. Chem. 266: 20833-20839 (1991)). Summaries can be found in textbooks of genetics and molecular biology, such as that by Hagemann (Allgemeine Genetik [General Genetics], Gustav Fischer Verlag, Stuttgart, 1986). Mutations may include transitions, transversions, insertions and deletions of at least one (1) base pair or nucleotide. Depending on the effect of the mutation on enzyme activity, missense mutations or to nonsense mutations may also be used. A missense mutation leads to the replacement of a given amino acid in a protein with a different, usually non-conservative amino acid. This usually impairs the function or activity of the protein and reduces it to a value of from 0 to 75%, 0 to 50%, 0 to 25%, 0 to 10% or 0 to 5%. A nonsense mutation leads to a stop codon in the coding region of the gene and thus to premature termination of translation. Insertions or deletions of at least one base pair in a gene lead to frame shift mutations which, in turn, result in incorrect amino acids being incorporated into the encoded protein or in the translation being prematurely terminated. If a stop codon is formed in the coding region as a consequence of the mutation, this also leads to translation being terminated prematurely. Deletions of at least one (1) or more codons typically also lead to a complete loss of the enzyme activity.

Directions for generating these mutations may be found in the prior art and can be obtained from textbooks of genetics and molecular biology such as the textbook by Knippers (Molekulare Genetik [Molecular Genetics], 6th edition, Georg Thieme Verlag, Stuttgart, Germany, 1995), Winnacker (Gene und Klone, [Genes and Clones], VHC Verlagsgesellschaft, Weinheim, Germany, 1990) or Hagemann (Allgemeine Genetik [General Genetics], Gustav Fischer Verlag, Stuttgart, 1986).

Mutations in genes can be incorporated into bacterial strains by means of gene or allele exchange. A customary method is that described by Hamilton et al. (J. Bacteriol. 171: 4617-4622 (1989)), using a conditionally replicating pSC101 derivative pMAK705. Other methods include that of Martinez-Morales et al. (J. Bacteriol. 181: 7143-7148 (1999)) or of Boyd et al. (J. Bacteriol. 182: 842-847 (2000)). It is also possible to transfer mutations to the relevant genes, or mutations which affect the expression of the relevant genes or open reading frames, by means of conjugation or transduction. It can also be advantageous, in addition to enhancing the open reading frame yaaU, to eliminate undesirable side-reactions (Nakayama: “Breeding of Amino Acid Producing Microorganisms”, in: Overproduction of Microbial Products, Krumphanzl, Sikyta, Vanek (eds.), Academic Press, London, UK, 1982).

The microorganisms which are prepared in accordance with the invention can be cultured in a batch process, in a fed-batch process, in a repeated fed-batch process or in a continuous process (DE102004028859.3 or U.S. Pat. No. 5,763,230). Culturing methods are summarized in the textbook by Chmiel (Bioprozesstechnik 1. Einführung in die Bioverfahrenstechnik [Bioprocess technology 1. Introduction to bioprocess technology], Gustav Fischer Verlag, Stuttgart, 1991)) or in the textbook by Storhas (Bioreaktoren und periphere Einrichtungen [Bioreactors and peripheral installations], Vieweg Verlag, Brunswick/Wiesbaden, 1994)). The culture medium used must satisfy the demands of the bacterial strains used. The American Society for Bacteriology manual “Manual of Methods for General Bacteriology” (Washington D.C., USA, 1981) contains descriptions of media for culturing a variety of microorganisms.

Sugars and carbohydrates, such as glucose, sucrose, lactose, fructose, maltose, molasses, starch and, where appropriate, cellulose, oils and fats, such as soybean oil, sunflower oil, peanut oil and coconut fat, fatty acids, such as palmitic acid, stearic acid and linoleic acid, alcohols, such as glycerol and ethanol, and organic acids, such as acetic acid, may be used as the carbon source. These substances may be used individually or as a mixture.

Organic nitrogen-containing compounds, such as peptones, yeast extract, meat extract, malt extract, corn steep liquor, soybean flour and urea, or inorganic compounds, such as ammonium sulfate, ammonium chloride, ammonium phosphate, ammonium carbonate and ammonium nitrate, may be used as the nitrogen source. The nitrogen sources may be used individually or as a mixture.

Phosphoric acid, potassium dihydrogen phosphate or dipotassium hydrogen phosphate, or the corresponding sodium-containing salts, may be used as the phosphorus source. In addition, the culture medium must contain salts of metals, such as magnesium sulfate or iron sulfate, which are required for growth. Finally, essential growth promoters, such as amino acids and vitamins, may be used. Suitable precursors can also be added to the culture medium. Ingredients may be added to the culture in the form of a single initial preparation or fed in during culture.

Fermentation is generally carried out at a pH of from 5.5 to 9.0, and preferably at 6.0 to 8.0. Basic compounds, such as sodium hydroxide, potassium hydroxide, ammonia or ammonia water, or acidic compounds, such as phosphoric acid or sulfuric acid, are used to control the pH of the culture. Antifoamants, such as fatty acid polyglycol esters, can be used to control foaming. Selectively acting substances, for example antibiotics, can be added to the medium in order to maintain the stability of plasmids. Oxygen or oxygen-containing gas mixtures, such as air, are passed into the culture in order to maintain aerobic conditions. The temperature of the culture is normally from 25° C. to 45° C. and preferably from 30° C. to 40° C. The culture process is continued until a maximum of L-amino acids or L-threonine has been formed. This objective is normally reached within 10 to 160 hours.

L-amino acids can be analyzed by means of anion exchange chromatography followed by derivatization with ninhydrin, as described in Spackman et al. (Anal. Chem. 30: 1190-1206 (1958)), or by means of reverse phase HPLC, so as described in Lindroth et al. (Anal. Chem. 51: 1167-1174 (1979)).

The process according to the invention can be used for fermentatively preparing L-amino acids, such as L-threonine, L-isoleucine, L-valine, L-methionine, L-homoserine, L-tryptophan and L-lysine, in particular L-threonine.

The following microorganism was deposited in the Deutsche Sammlung für Mikroorganismen und Zellkulturen [German collection of microorganisms and cell cultures] (DSMZ, Brunswick, Germany) in accordance with the Budapest Treaty:
Escherichia coli strain E. coli MG442 as DSM 16574.

The present invention is explained in more detail below with the aid of implementation examples.

### EXAMPLES

Minimal (M9) and complete (LB) media used for Escherichia coli are described by J. H. Miller (A short course in bacterial genetics, Cold Spring Harbor Laboratory Press (1992)). The isolation of plasmid DNA from Escherichia coli, and also all techniques for restricting, ligating and treating with Klenow phosphatase and alkali phosphatase, are carried out as described in Sambrook et al. (Molecular Cloning—A Laboratory Manual Cold Spring Harbor Laboratory Press (1989)). Unless otherwise indicated, Escherichia coli are transformed as described in Chung et al. (Proc. Nat'l Acad. Sci. USA 86: 2172-2175 (1989)). The incubation temperature when preparing strains and transformants is 37° C.

### Example 1

### Constructing the Expression Plasmid pTrc99AyaaU

The E. coli K12 yaaU ORF is amplified using the polymerase chain reaction (PCR) and synthetic oligonucleotides. PCR primers are synthesized (MWG Biotech, Ebersberg, Deutschland) on the basis of the nucleotide sequence of the yaaU ORF in E. coli K12 MG1655 (Accession Number AE000114), Blattner et al. (Science 277: 1453-1474 (1997)). The sequences of the primers are modified so as to form recognition sites for restriction enzymes. The EcoRI recognition sequence is selected for the yaaU-ex₁ primer and the BamHI recognition sequence is selected for the yaaU-ex2 primer, with these sequences being underlined in the nucleotide sequences shown below:

yaaU-ex1:

(SEQ ID NO:1)

5′ -GATCTGAATTCTAAGGAATAACCATGCAACCGTC- 3′
yaaU-ex2:

(SEQ ID NO:2)

5′ -GATCTAGGATCCCAATTTACCCCATTCTCTGC- 3′

The E. coli K12 MG1655 chromosomal DNA used for PCR is isolated using “Qiagen Genomic-tips 100/G” (QIAGEN, Hilden, Germany) in accordance with the manufacturer's instructions. A DNA fragment of approx. 1371 bp in size (SEQ ID NO:3) can be amplified under standard PCR conditions (Innis et al., PCR Protocols. A Guide to Methods and Applications, Academic Press (1990)) using Vent DNA polymerase (New England Biolaps GmbH, Frankfurt, Germany) and the specific primers.

The amplified yaaU fragment is ligated to the vector pCR-Blunt II-TOPO (Zero TOPO TA Cloning Kit, Invitrogen, Groningen, Netherlands) in accordance with the manufacturer's instructions and transformed into the E. coli strain TOP10. Plasmid-harboring cells are selected on LB Agar containing 50 μg of kanamycin/ml. After the plasmid DNA has been isolated, the vector is cleaved with the enzymes EcoRV and EcoRI and, after the cleavage has been checked in a 0.8% agarose gel, is designated pCRBluntyaaU.

The vector pCRBluntyaaU is cleaved with the enzymes EcoRI and BamHI and the yaaU fragment is separated in a 0.8% agarose gel. It is then isolated from the gel (QIAquick Gel Extraction Kit, QIAGEN, Hilden, Germany) and ligated to the vector pTrc99A (Pharmacia Biotech, Uppsala, Sweden) which has been digested with the enzymes BamHI and EcoRI. The E. coli strain XL1Blue MRF′ (Stratagene, La Jolla, USA) is transformed with the ligation mixture and plasmid-harboring cells are selected on LB agar containing 50 μg of ampicillin/ml.

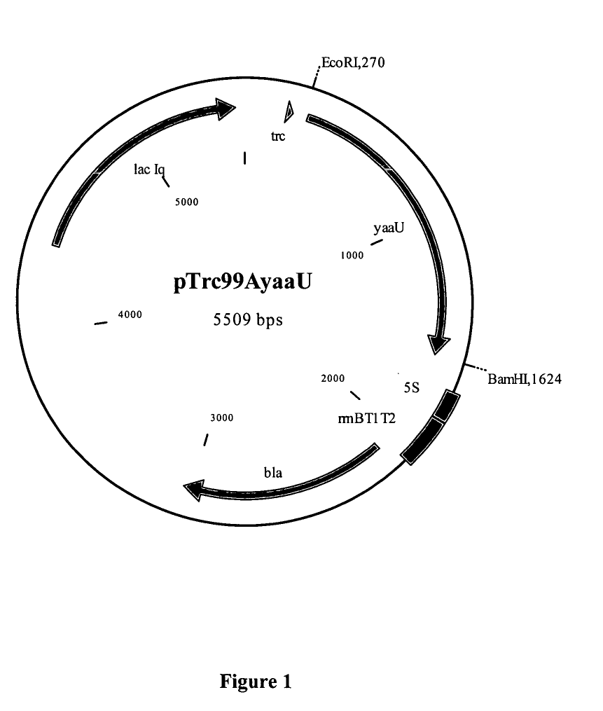

That cloning has been successful can be demonstrated, after the plasmid DNA has been isolated, by performing a control cleavage using the enzymes EcoRI/BamHI and EcoRV. The plasmid is designated pTrc99AyaaU (FIG. 1).

### Example 2

### Preparing L-Threonine Using the Strain MG442/pTrc99AyaaU

The L-threonine-producing E. coli strain MG442 is described in U.S. Pat. No. 4,278,765 and is deposited in the Russian national collection of industrial microorganisms (VKPM, Moscow, Russia) as CMIM B-1628 and in the Deutsche Sammlung für Mikroorganismen und Zellkulturen [German collection of microorganisms and cell cultures] (DSMZ, Brunswick, Germany), in accordance with the Budapest Treaty, as DSM 16574.)

The strain MG442 is transformed with the expression plasmid pTrc99AyaaU described in Example 1, and with the vector pTrc99A, and plasmid-harboring cells are selected on LB agar containing 50 μg of ampicillin/ml. This results in the strains MG442/pTrc99AyaaU and MG442/pTrc99A. Selected individual colonies are then propagated further on minimal medium having the following composition: 3.5 g of Na₂HPO₄*2H₂O/l, 1.5 g of KH₂PO₄/l, 1 g of NH₄Cl/l, 0.1 g of MgSO₄*7H₂O/l, 2 g of glucose/l, 20 g of agar/l, 50 mg of ampicillin/l.

The formation of L-threonine is checked in 10 ml batch cultures which are contained in 100 ml Erlenmeyer flasks. For this, a 10 ml preculture medium of the following composition: 2 g of yeast extract/l, 10 g of (NH₄)₂SO₄/l, 1 g of KH₂PO₄/l, 0.5 g of MgSO₄*7H₂O/l, 15 g of CaCO₃/l, 20 g of glucose/l, 50 mg of ampicillin/l, is inoculated and incubated at 37° C. and 180 rpm for 16 hours on a Kühner AG ESR incubator (Birsfelden, Switzerland). In each case 250 μl of this preliminary culture are inoculated into 10 ml of production medium (25 g of (NH₄)₂SO₄/l, 2 g of KH₂PO₄/l, 1 g of MgSO₄.7H₂O/l, 0.03 g of FeSO₄*7H₂O/l, 0.018 g of MnSO₄*1H₂O/l, 30 g of CaCO₃/l, 20 g of glucose/l, 50 mg of ampicillin/l) and incubated at 37° C. for 48 hours. The formation of L-threonine by the starting strain MG442 is checked in the same way with, however, no ampicillin being added to the medium. After the incubation, the optical density (OD) of the culture suspension is determined at a measurement wavelength of 660 nm using a Dr. Lange LP2W photometer (Düsseldorf, Germany).

An Eppendorf-BioTronik amino acid analyzer (Hamburg, Germany) is then used to determine, by means of ion exchange chromatography and post-column reaction involving ninhydrin detection, the concentration of the resulting L-threonine in the culture supernatant, which has been sterilized by filtration. The result of the experiment is shown in table 1.

TABLE 1

| Strain | OD (660 nm) | L-Threonine g/l |
|---|---|---|
| MG442 | 5.6 | 1.4 |
| MG442/pTrc99A | 3 | 1.3 |
| MG442/pTrc99AyaaU | 4.1 | 2.7 |

### Abbreviations

Length specifications are to be regarded as being approximate. The abbreviations and designations employed have the following meanings:
bla: gene which encodes resistance to ampicillin
lac Iq: gene for the trc promoter repressor protein
trc: trc promoter region, IPTG-inducible
yaaU: coding region of the yaaU gene
5S: 5S rRNA region
rrnBT: rRNA terminator region

The abbreviations for the restriction enzymes have the following meaning:
BamHI: restriction endonuclease from Bacillus amyloliquefaciens H
EcoRI: restriction endonuclease from Escherichia coli RY13
EcoRV: restriction endonuclease from Escherichia coli B946

All references cited herein are fully incorporated by reference. Having now fully described the invention, it will be understood by those of skill in the art that the invention may be practiced within a wide and equivalent range of conditions, parameters and the like, without affecting the spirit or scope of the invention or any embodiment thereof.

1. A recombinant microorganism of the Enterobacteriaceae family comprising an enhanced or overexpressed yaaU ORF.

2. A recombinant microorganism of claim 1, wherein said yaaU ORF comprises a polynucleotide encoding a polypeptide with an amino acid sequence that is at least 90% identical to an amino acid sequence selected from the group consisting of: SEQ ID NO:4, SEQ ID NO:6 and SEQ ID NO:8 and wherein said polynucleotide is enhanced.

3. A recombinant microorganism comprising an overexpressed or enhanced polynucleotide which corresponds to the yaaU ORF and which is selected from the group consisting of:
a) a polynucleotide comprising the nucleotide sequence of SEQ ID NO:3, SEQ ID NO:5 or SEQ ID NO:7;
b) a polynucleotide comprising the nucleotide sequence of SEQ ID NO:3, SEQ ID NO:5 or SEQ ID NO:7 within the limits of the degeneracy of the genetic code;
c) a polynucleotide comprising a sequence which hybridizes, under stringent conditions, with a sequence that is complementary to the sequence of SEQ ID NO:3, SEQ ID NO:5 or SEQ ID NO:7;
d) a polynucleotide comprising the sequence of SEQ ID NO:3, SEQ ID NO:5 or SEQ ID NO:7 which contains one or more functionally neutral sense mutants.

4. The recombinant microorganism of claim 2, wherein said polypeptide has an amino acid sequence that is at least 95% identical to a sequence selected from the group consisting of: SEQ ID NO:4, SEQ ID NO:6 and SEQ ID NO:8.

5. The recombinant microorganism of claim 2, wherein said polypeptide has an amino acid sequence that is at least 95% identical to a sequence selected from the group consisting of: SEQ ID NO:4, SEQ ID NO:6 and SEQ ID NO:8.

6. The recombinant microorganism of any one of claims 1 to 5, wherein said recombinant microorganism is produced by a process comprising the transformation, transduction, or conjugation, of a nonrecombinant parent microorganism with a vector comprising the yaaU ORF, an allele of this ORF, and/or a promotor.

7. The recombinant microorganism of claim 1, wherein the copy number of the yaaU ORF or the allele of this ORF has been increased by at least 1.

8. The recombinant microorganism of claim 7, wherein the increase in said copy number is achieved by integrating said ORF or said allele into the chromosome of the microorganism.

9. The recombinant microorganism of claim 7, wherein the increase in said copy number is achieved by means of a vector which replicates extrachromosomally.

10. The recombinant microorganism of either claim 1 or 2, wherein said polynucleotide is enhanced by a process comprising either:
a) mutating the promoter and regulatory region or the ribosomal binding site upstream of the yaaU ORF; or
b) incorporating expression cassettes or promoters upstream of the yaaU ORF.

11. The recombinant microorganism of either claim 1 or 2, wherein said yaaU ORF is under the control of a promoter enhancing the expression of the ORF.

12. The recombinant microorganism of any one of claims 1-5 or 7-9, wherein the concentration or activity of the yaaU protein is increased by at least 10% relative to the activity or concentration of the yaaU protein in the nonrecombinant parent microorganism.

13. The recombinant microorganism of any one of claims 1-5 or 7-9, futher comprising the overexpression of at least one gene of a metabolic pathway for the biosynthesis of an L-amino acid.

14. The recombinant microorganism of claim 13, wherein said recombinant microorganism is in a genus selected from the group consisting of: Escherichia; Erwinia; Providencia; and Serratia.

15. The recombinant microorganism of claim 13 or 14, wherein said amino acid is L-threonine.

16. A process for preparing an L-amino acid by fermentation comprising:
a) culturing the recombinant microorganism of any one of claims 1-5 or 7-9 in a medium under conditions under which said L-amino acid is enriched in the medium or in the cells, and
b) after step a), isolating said L-amino acid with from 0 to 100% of the constituents of the fermentation broth, and/or the biomass, remaining in the isolated product.

17. The process of claim 16, wherein said recombinant microorganism is in a genus selected from the group consisting of: Escherichia; Erwinia; Providencia; and Serratia.

18. The process of claim 17, wherein said L-amino acid is L-threonine.

19. The process of claim 17, wherein said L-amino acid is selected from the group consisting of: L-asparagine; L-serine; L-glutamate; L-glycine; L-alanine; L-cysteine; L-valine; L-methionine; L-proline; L-isoleucine; L-leucine; L-tyrosine; L-phenylalanine; L-histidine; L-lysine; L-tryptophan; L-arginine; and L-homoserine.

20. The process of claim 19, wherein said L-amino acid is selected from the group consisting of: L-isoleucine; L-valine; L-methionine; L-homoserine; L-tryptophan; and L-lysine.

21. The process of claim 16, wherein said recombinant microorganism overexpresses more genes selected from the group consisting of:
a) at least one gene of the thrABC operon encoding aspartate kinase, homoserine dehydrogenase, homoserine kinase and threonine synthase;
b) the pyruvate carboxylase-encoding Corynebacterium glutamicum pyc gene;
c) the phosphoenolpyruvate synthase-encoding pps gene,
d) the phosphoenolpyruvate carboxylase-encoding ppc gene;
e) the pntA or pntB genes encoding the subunits of pyridine transhydrogenase;
f) the rhtB gene encoding the homoserine resistance-mediating protein;
g) the rhtC gene encoding the threonine resistance-mediating protein;
h) the threonine export carrier protein-encoding Corynebacterium glutamicum thrE gene;
i) the glutamate dehydrogenase-encoding gdhA gene;
j) the phosphoglucomutase-encoding pgm gene;
k) the fructose biphosphate aldolase-encoding fba gene;
l) the ptsH gene encoding the phosphohistidine protein hexose phosphotransferase;
m) the ptsI gene encoding enzyme I of the phosphotransferase system;
n) the crr gene encoding the glucose-specific IIA component;
o) the ptsG gene encoding the glucose-specific IIBC component;
p) the lrp gene encoding the regulator of the leucine regulon;
q) the fadR gene encoding the regulator of the fad regulon;
r) the iclR gene encoding the regulator of central intermediary metabolism;
s) the ahpC gene encoding the small subunit of alkyl hydroperoxide reductase;
t) the ahpF gene encoding the large subunit of alkyl hydroperoxide reductase;
u) the cysteine synthase A-encoding cysK gene;
v) the cysB gene encoding the regulator of the cys regulon;
w) the cysJ gene encoding the NADPH sulfite reductase flavoprotein;
x) the cysI gene encoding the NADPH sulfite reductase hemoprotein;
y) the adenylyl sulfate reductase-encoding cysH gene;
z) the rseA gene encoding a membrane protein which possesses anti-sigmaE activity;
aa) the rseC gene encoding a global regulator of the sigmaE factor;
bb) the sucA gene encoding the decarboxylase subunit of 2-ketoglutarate dehydrogenase;
cc) the sucB gene encoding the dihydrolipoyltranssuccinase E2 subunit of 2-ketoglutarate dehydrogenase;
dd) the sucC gene encoding the α-subunit of succinyl-CoA synthetase;
ee) the sucD gene encoding the α-subunit of succinyl-CoA synthetase;
ff) the aceE gene encoding the E1 component of the pyruvate dehydrogenase complex;
gg) the aceF gene encoding the E2 component of the pyruvate dehydrogenase complex;
hh) the rseB gene encoding the regulator of the SigmaE factor activity; and
ii) the gene product of the Escherichia coli yodA open reading frame (ORF).

22. The process of claim 21, wherein said recombinant microorganism is in a genus selected from the group consisting of: Escherichia; Erwinia; Providencia; and Serratia.

23. The process of claim 22, wherein said L-amino acid is L-threonine.

24. The process of claim 22, wherein said L-amino acid is selected from the group consisting of: L-asparagine; L-serine; L-glutamate; L-glycine; L-alanine; L-cysteine; L-valine; L-methionine; L-proline; L-isoleucine; L-leucine; L-tyrosine; L-phenylalanine; L-histidine; L-lysine; L-tryptophan; L-arginine; and L-homoserine.

25. The process of claim 24, wherein said L-amino acid is selected from the group consisting of: L-isoleucine; L-valine; L-methionine; L-homoserine; L-tryptophan; and L-lysine.

26. The process of claim 16 wherein said recombinant microorganism comprises a metabolic pathway which reduces the formation of said L-amino acid that is at least partially attenuated.

27. The process of claim 26, wherein said metabolic pathway is attenuated by reducing or eliminating the expression of one or more genes selected from the group consisting of:
a) the threonine dehydrogenase-encoding tdh gene;
b) the malate dehydrogenase-encoding mdh gene;
c) the gene product of the Escherichia coli yjfA open reading frame (ORF);
d) the gene product of the Escherichia coli ytfP open reading frame (ORF);
e) the pckA gene encoding phosphoenolpyruvate carboxykinase;
f) the pyruvate oxidase-encoding poxB gene;
g) the dgsA gene encoding the DgsA regulator of the phosphotransferase system;
h) the fruR gene encoding the fructose repressor;
i) the rpoS gene encoding the sigma³⁸ factor; and
j) the aspartate ammonium lyase-encoding aspA gene.

28. The process of claim 27, wherein said recombinant microorganism is in a genus selected from the group consisting of: Escherichia; Erwinia; Providencia; and Serratia.

29. The process of claim 28, wherein said L-amino acid is L-threonine.

30. The process of claim 28, wherein said L-amino acid is selected from the group consisting of: L-asparagine; L-serine; L-glutamate; L-glycine; L-alanine; L-cysteine; L-valine; L-methionine; L-proline; L-isoleucine; L-leucine; L-tyrosine; L-phenylalanine; L-histidine; L-lysine; L-tryptophan; L-arginine; and L-homoserine.

31. The process of claim 30, wherein said L-amino acid is selected from the group consisting of: L-isoleucine; L-valine; L-methionine; L-homoserine; L-tryptophan; and L-lysine.


SEQUENCE LISTING (8 sequences)

SEQ ID NO: 1 - DNA, 34 bp, Artificial sequence
  Primer (1)..(34): yaaU-ex1
gatctgaattctaaggaataaccatgcaaccgtc

SEQ ID NO: 2 - DNA, 32 bp, Artificial sequence
  Primer (1)..(32): yaaU-ex2
gatctaggatcccaatttaccccattctctgc

SEQ ID NO: 3 - DNA, 1372 bp, Escherichia coli
  PCR-product (1)..(1372): yaaU PCR-product
gatctgaattcttaaggaataaccatgcaaccgtccagaaactttgacgatctcaaattctcctctattcaccgccgcattttgctgtggggaagcggtggtccgtttctggatggttatgtactggtaatgattggcgtggcgctggagcaactgacgccggcgctgaaactggacgctgactggattggcttgctgggcgcgggaacgctcgccgggctgttcgttggcacatcgctgtttggttatatttccgataaagtcggacggcgcaaaatgttcctcattgatatcatcgccatcggcgtgatatcggtggcgacgatgtttgtttcatcccccgtcgaactgttggtgatgcgggtacttatcggcattgtcatcggtgcagattatcccatcgccacctcaatgatcaccgagttctccagtacccgtcagcgggcgttttccatcagctttattgccgcgatgtggtatgtcggcgcgacctgtgccgatctggtcggctactggctttatgatgtggaaggcggctggcgctggatgctgggtagcgcggcgatcccctgtttgttgattttgattggtcgattcgaactgcctgaatctccccgctggttattacgcaaagggcgagtaaaagagtgcgaagagatgatgatcaaactgtttggcgaaccggtggctttcgatgaagagcagccgcagcaaacccgttttcgcgatctgtttaatcgccgccattttccttttgttctgtttgttgccgccatctggacctgccaggtgatcccaatgttcgccatttacacctttggcccgcaaatcgttggtttgttgggattgggggttggcaaaaacgcggcactagggaatgtggtgattagcctgttctttatgctcggctgtattccgccgatgctgtggttaaacactgccggacggcgtccattgttgattggcagctttgccatgatgacgctggcgctggcggttttggggctaatcccggatatggggatctggctggtagtgatggcctttgcggtgtatgcctttttctctggcgggccgggtaatttgcagtggctctatcctaatgaactcttcccgacagatatccgcgcctctgccgtgggcgtgattatgtccttaagtcgtattggcaccattgtttcgacctgggcactaccgatctttatcaataattacggtatcagtaacacgatgctaatgggggcgggtatctcgctgtttggcttgttgatttccgtagcgtttgccccggagactcgagggatgtcactggcgcagaccagcaatatgacgatccgcgggcagagaatggggtaaattgggatcctagatc

SEQ ID NO: 4 - protein, 443 aa, Escherichia coli
MQPSRNFDDLKFSSIHRRILLWGSGGPFLDGYVLVMIGVALEQLTPALKLDADWIGLLGAGTLAGLFVGTSLFGYISDKVGRRKMFLIDIIAIGVISVATMFVSSPVELLVMRVLIGIVIGADYPIATSMITEFSSTRQRAFSISFIAAMWYVGATCADLVGYWLYDVEGGWRWMLGSAAIPCLLILIGRFELPESPRWLLRKGRVKECEEMMIKLFGEPVAFDEEQPQQTRFRDLFNRRHFPFVLFVAAIWTCQVIPMFAIYTFGPQIVGLLGLGVGKNAALGNVVISLFFMLGCIPPMLWLNTAGRRPLLIGSFAMMTLALAVLGLIPDMGIWLVVMAFAVYAFFSGGPGNLQWLYPNELFPTDIRASAVGVIMSLSRIGTIVSTWALPIFINNYGISNTMLMGAGISLFGLLISVAFAPETRGMSLAQTSNMTIRGQRMG

SEQ ID NO: 5 - DNA, 1395 bp, Shigella flexneri
  CDS (1)..(1395): yaaU coding region
atgcaaccgtccagaaactttgacgatctcaaattctcctctattcaccgccgcattttgctgtggggaagcggtggtccgtttctggatggttatgtactggtaatgattggcgtggcgctggagcaactgactccggcgctgaaactggacgctgactggattggcttgctgggcgcgggaacgctcgccgggctgttcgttggcacatcgctgtttggctatatctccgataaagtcggacggcgcaaaatgttcctcattgatatcatcgccatcggcgtgatatcggtggcgacgatgtgtgtttcatcccccgtcgaactgttggtgatgcgggtactcatcggcattgtcatcggtgcagattatcccatcgccacttcgatgatcaccgagttctccagtacccgtcagcgggcgttttccatcagctttattgccgcgatgtggtatgtcggcgcgacctgcgccgatctggtcggctactggctttatgatgtggaaggtggttggcgctggatgctgggtagcgcggcgatcccttgtttgttgattttgattggtcgattcgaactgccagaatctccccgctggttattacgcaaagggcgagtaaaagagtgcgaagagatgatgataaaactgtttggcgaaccggtggctttcgatgaagagcagccgcagcaaacccgttttcgcgatctgtttaatcgccgccattttccttttgttctgtttgttgccgccatctggacctgccaggtgatcccaatgttcgccatttacacctttggcccgcaaatcgttggttcgttgggattgggggttggcaaaaacgcggcactagggaatgtggtgattagcctgttctttatgctcggctgtattccgccgatgctgtggctaaacactgccggacggcgtccattgttgattggcagctttgccatgatgacgctggcgttggcggttttggggctaatcccggatatggggatctggctggtagtgatggcctttgcggtgtatgcctttttctctggcgggccgggtaatttgcagtggctctatcctaatgaactcttcccgacggatatccgcgcctctgccgtgggcgtgattatgtccttaagtcgtattggcaccattgtttcgacctgggcactgccgatatttatcaataattacggtatcagtaacacgatgctaatgggggcgggtatctcgctgtttggcttgttgatttccgtagcgtttgccccggagactcgaggaatgtcactggcgcagaccagcaatatgacgatccgcgggcagagaatgggattatttgttcagatttctctcttttctgaatcaatattattgactataagccgcgtgaatatatga

SEQ ID NO: 6 - protein, 464 aa, Shigella flexneri
MQPSRNFDDLKFSSIHRRILLWGSGGPFLDGYVLVMIGVALEQLTPALKLDADWIGLLGAGTLAGLFVGTSLFGYISDKVGRRKMFLIDIIAIGVISVATMCVSSPVELLVMRVLIGIVIGADYPIATSMITEFSSTRQRAFSISFIAAMWYVGATCADLVGYWLYDVEGGWRWMLGSAAIPCLLILIGRFELPESPRWLLRKGRVKECEEMMIKLFGEPVAFDEEQPQQTRFRDLFNRRHFPFVLFVAAIWTCQVIPMFAIYTFGPQIVGSLGLGVGKNAALGNVVISLFFMLGCIPPMLWLNTAGRRPLLIGSFAMMTLALAVLGLIPDMGIWLVVMAFAVYAFFSGGPGNLQWLYPNELFPTDIRASAVGVIMSLSRIGTIVSTWALPIFINNYGISNTMLMGAGISLFGLLISVAFAPETRGMSLAQTSNMTIRGQRMGLFVQISLFSESILLTISRVNI

SEQ ID NO: 7 - DNA, 1341 bp, Salmonella typhimurium
  CDS (1)..(1341): yaaU coding region
atgcagcagcccaggaactttgatgaccttaaattctcctccattcatcgcagaattatgctgtggggaagcggcggcccgttccttgatggctacgtgctggtgatcattggcgtggcgctggaacaactgacgccgctattgcaccttgatgctgaatggattggcgcgctcggcgcggctacgcttgccggactgtttatcggcacctcgttgtttggctatatttgcgataaagtcgggcggcgtaaaatgttcctgcttgatattatcgctatcggcgttatctccgtggcgaccatgtttgtttccacgccgcttgaattactggtgatgcgcgttttaattggcattgttatcggcgcggattaccctatcgctacttccatgattacggagttttccaacacccgccagcgcgcgttctccatcggttttatcgccgcgatgtggtacgtcggcgcgacctgcgccaacctggtgggctactggctgtatgacatggaaggcggctggcgttggatgcttggcagcgcctttatcccgtgcttaatcattttgattggccgctttgacctgccggaatcgccacgctggctattgcgaaaaggacgggtaaaagagtgcgaacaaatgatgatcaaactgttcggcgaaccggtctgctttgacgatgagctgccgcaggagacccgcttcctgcagctgtttaaccgccgtcatttcccgtttgtactgttcgttgcggctatctggacctgtcaggtgatcccgatgttcgctatctatacctttgggccgcagatcgtcgggttactcggctgggagcaggggcgcaacgcggcgcttggcaatgtggtgattagcctcttttttatgctgggctgtataccggcgatgttctggctaaacagtattggccgtcgccctctgcttattggcagcttcgccatgatgactatcgcgctggcgctgctggggctggtgtcaaatctgggcatcatactggtcgtggtggcgtttgcggtatacgccttcttttccggcggaccgggcatactgcaatggctttatcctaatgaactcttcccgacggatattcgcgcttcggccgtgggggtcatcatgtcattaagccgcatcggaaccattgtctccacctgggcattaccgatttttattactcgttacggtattaataacgtcatgctgattggcgcgttgatttcgttagtcgggcttggcgtatcggtgatgtttgccccggaaacccgcggcttgacgctaacacagaccggaaacatgactttgcgcggaacgccatcggataaccctcgttaa

SEQ ID NO: 8 - protein, 446 aa, Salmonella typhimurium
MQQPRNFDDLKFSSIHRRIMLWGSGGPFLDGYVLVIIGVALEQLTPLLHLDAEWIGALGAATLAGLFIGTSLFGYICDKVGRRKMFLLDIIAIGVISVATMFVSTPLELLVMRVLIGIVIGADYPIATSMITEFSNTRQRAFSIGFIAAMWYVGATCANLVGYWLYDMEGGWRWMLGSAFIPCLIILIGRFDLPESPRWLLRKGRVKECEQMMIKLFGEPVCFDDELPQETRFLQLFNRRHFPFVLFVAAIWTCQVIPMFAIYTFGPQIVGLLGWEQGRNAALGNVVISLFFMLGCIPAMFWLNSIGRRPLLIGSFAMMTIALALLGLVSNLGIILVVVAFAVYAFFSGGPGILQWLYPNELFPTDIRASAVGVIMSLSRIGTIVSTWALPIFITRYGINNVMLIGALISLVGLGVSVMFAPETRGLTLTQTGNMTLRGTPSDNPR